(19) United States
(12) Patent Application Publication
(10) Pub. No.: US 2018/0162790 A1
(43) Pub. Date: Jun. 14, 2018

(54) MODIFIED PROPANE DEHYDROGENATION SYSTEM AND METHOD FOR PRODUCING ONE OR MORE CHEMICAL PRODUCTS FROM PROPANE

(71) Applicants: ThyssenKrupp Industrial Solutions AG, Essen (DE); ThyssenKrupp AG, Essen (DE)

(72) Inventor: Muhamad Fouad, Centennial, CO (US)

(73) Assignees: ThyssenKrupp Industrial Solutions AG, Essen (DE); ThyssenKrupp AG, Essen (DE)

(21) Appl. No.: 15/375,085

(22) Filed: Dec. 11, 2016

(51) Int. Cl. C07C 5/327; C07C 253/18; C07C 51/15; C07C 29/151; C01C 1/04; B01J 19/24

(52) U.S. Cl. CPC C07C 5/327; C07C 253/18; C07C 51/15; B01J 2219/24; C01C 1/0405; B01J 19/24; B01J 19/245; C07C 29/1518

(57) ABSTRACT

Disclosed is a modified propene dehydrogenation (PDH) system and method for producing one or more chemical products from propane as the sole feed stock. The modified PDH system includes a reactor for converting propane into a stream of propene, hydrogen, and waste gas. It further includes a de-ethanizer stripper downstream of the PDH reactor for separating the reactor output gas into a stream of propene as one of an end product or an intermediate product, and a stream of hydrogen and waste gas. The modified PDH system also includes a hydrogen recovery unit disposed downstream of the de-ethanizer stripper system for separating the stream of waste gas and hydrogen into separate streams of waste gas, and hydrogen, with the hydrogen stream being one of an end product or an intermediate product. The modified PDH system can produce propene, hydrogen, ammonia, acrylonitrile, urea, or methanol.

### FIELD

This disclosure relates to a modified propane dehydrogenation (PDH) system and process, which is configured to produce propene and any one of hydrogen, ammonia from the hydrogen, acrylonitrile from the ammonia, and/or additional alternate products, all from propane as a sole feed stock. The present disclosure further relates to a system and method for utilization of propene produced by a modified PDH system and process, in conjunction with a least one additional product also produced in the same modified PDH system and process, in order to produce either ammonia or acrylonitrile. The present disclosure further relates to the synergistic use of at least two sub systems and sub processes within the modified PDH process.

### BACKGROUND

Propene (C₃H₆), also commonly known as propylene, is an important chemical intermediate chemical product. It can be used to produce numerous polymers. Around two thirds of the global demand for propene is consumed to produce polypropylene, which is in turn used, for example, to make plastic films and packing materials, or is used in the electrical and/or automobile industries, for example. The second-largest use of propene is in the production of acrylonitrile, with the third largest use of propene being in the production of propylene oxide. The industrial demand for propene is increasing, so producing propene is of great commercial interest for chemical companies.

Propene (or propylene) as an end product can either be produced from crude oil feed stock in refineries (which accounts for about 80% of all global propene/propylene production), or may be produced from propane (C₃H₈) feed stock in a conventional propane dehydrogenation (PDH) plant by an associated PDH process (which accounts for about 10% of all global propene/propylene production). For production of propene in a PDH system or plant, despite the relatively simple chemistry involved in the PDH process, industrial implementation of conventional PDH systems or plants is very complicated due to side reactions that occur, such as deep dehydrogenation, hydogenolysis, cracking, polymerization and coke formation. Side reactions impact the complexity of the process, operating costs and the purity of the products. The conventional PDH system and process converts propane into propene and reaction byproducts, including hydrogen, which byproducts are usually used as fuel that is burned to drive the PDH reaction itself. As a result, propene tends to be the only end product produced from the operation of a conventional PDH system and process. However, due to the rising exploitation of shale gas, the purchase price of propane (C₃H₈), which is the sole feed stock for the PDH process, has decreased over the last few years making it an attractive feed stock to use to produce propene (C₃H₆) and/or any other products capable of being made from propane (C₃H₈). Chemical companies are already planning to establish PDH plants in at least the United States to take advantage of this low price raw material obtained from shale gas.

In addition, another chemical product, Acrylonitrile, is one of the leading chemicals produced worldwide, with a worldwide production of about 6 million tonnes (or metric tons) in 2003. The most important applications and uses of acrylonitrile are in the production of acrylic fibers, thermoplastics such as styrene acrylonitrile (SAN) and acrylonitrile butadiene styrene (ABS), technical rubbers, adiponitrile, and various specialty polymers. Currently there are two main processes that may be used to make acrylonitrile, typically in stand-alone plants; (1) propene (or propylene) ammoxidation (see FIG. 1a), and (2) propane ammoxidation (see FIG. 1b) which is currently only being practiced in one plant globally. Both systems/processes have advantages and disadvantages.

Referring to FIG. 1a, a high-level schematic diagram of the system/process for producing acrylonitrile by the known process of propene (or propylene) ammoxidation is shown. Of the two methods discussed for producing acrylonitrile, about 90% of the worldwide acrylonitrile is produced by propene (or propylene) ammoxidation. Here, it is shown that to produce acrylonitrile by this process, the system/process typically uses crude oil that is processed in an oil refinery to produce propene (or propylene) as the first required feed stock, and natural gas that is reacted in a separate ammonia plant to produce ammonia as the second required feed stock. The propene and ammonia are each then transported (e.g. typically by costly trains or semi-trucks) to the acrylonitrile plant to be used together as feed stock to produce acrylonitrile by the propene (or propylene) ammoxidation process. The advantages of this process for producing acrylonitrile are that it can achieve high acrylonitrile yields (e.g. 81%) with a minimum required amount of ammonia and a minimum formation of byproducts. The disadvantages are that (a) the price of the propene feed stock made from oil is high, (b) there is significant price volatility with respect to both propene and ammonia (because they are both made from oil and natural gas respectively, which themselves also have significant price volatility), and (c) the price of ammonia and propene feed stocks have associated transportation costs that must be factored in to their purchase price, and which transportation costs also fluctuate with the price of oil.

Referring to FIG. 1b, a high-level schematic diagram of the system/process for producing acrylonitrile by the process of propane ammoxidation is shown. Here, it is shown that to produce acrylonitrile by this process, the system/process typically uses propane (e.g. that has been obtained from shale gas) as the first required feed stock, and natural gas that is reacted in an ammonia plant to produce ammonia as the second needed feed stock. The propane and ammonia are each then transported (e.g. by costly trains or semi-trucks) to the acrylonitrile plant to produce acrylonitrile by the propane ammoxidation process. The advantages of this process for producing acrylonitrile are that it uses cheap propane (C₃H₈) feed stock (typically from abundant shale gas supplies in the United States), and there is an excess supply of propane readily available. The disadvantages are that this process results in a considerably low acrylonitrile yield (e.g. less than 59%) with a high amount of byproduct formation and a high amount of ammonia consumption. It also results in a lower single pass conversion rate and higher amount of CO₂ emissions than in the propene (or propylene) ammoxidation process depicted in FIG. 1a.

Furthermore, production of ammonia is one of the most technologically complex and energy intensive industrial processes. In the conventional production of chemicals like urea, acrylonitrile, and other chemicals that require ammonia as a feed stock, the ammonia production itself accounts for approximately 80% of the total energy required for production of urea, acrylonitrile and any such other chemicals. As mentioned previously, currently both conventional PDH systems or plants and ammonia plants are independently implemented as separate stand-alone facilities for production of propene and ammonia respectively.

Thus, with the above background regarding propene and acrylonitrile production systems and methods, together with an understanding of the advantages and the disadvantages associated with each conventional system for their respective production, there exists a need for a new system and process that is capable of producing propene as well as another valuable intermediary or end product form a single process, using only propane as the sole feed stock. There also exists a need for a system that is capable of producing acrylonitrile that has the benefits of both of the previous acrylonitrile production processes (propene ammoxidation and propane ammoxidation), but without the associated disadvantages thereof. There is also a need for a system and process that can take advantage of synergies between a propene production system and additional downstream systems to produce propene and one or more valuable additional intermediary and/or end products, such as for example ammonia, methanol, urea, or acrylonitrile. There is also further a need for a system and process that can take advantage of efficiencies between a PDH system and process and additional downstream systems to produce propene and one or more valuable additional intermediary and/or end products in a greener/cleaner, cheaper, and more energy efficient manner than has been done in the past with stand-alone systems.

### SUMMARY

A modified propane dehydrogenation (PDH) system or plant of the present disclosure is configured to increase propane utilization efficiency in the PDH system or plant and process as well as ensure the efficient use of any product or byproduct from the modified PDH system or plant and its process.

As disclosed herein, a modified PDH plant or system is configured to produce propene by a modified PDH process, and includes a hydrogen (or dihydrogen (H₂)) recovery unit configured to recover hydrogen from the PDH system, and convey a hydrogen stream from the hydrogen recovery unit to at least one additional downstream plant or system, for example an ammonia production system, an acrylonitrile production system, a urea production system, a methanol production system, or any other system or plant that may require hydrogen as a reactant. In the present disclosure, the terms hydrogen and dihydrogen may be used interchangeably and should be understood to be synonyms of each other.

The hydrogen recovery unit may provide for high efficiency and/or synergetic use of process fluids or process gasses. In particular, it has been found that many different favorable configurations or interconnections of different plants/systems may be realized if hydrogen in the waste gas stream is not used as fuel to be burned to operate the PDH system, but is instead removed from the waste gas stream and used as a reactant for additional processes downstream in a modified PDH system or process. In one aspect of the present disclosure, hydrogen produced by a modified PDH process of the present disclosure may be used as make-up gas fed to the front-end of an ammonia production system to produce an ammonia product. In this manner, eliminating the hydrogen production catalyst devices from ammonia manufacturing plants or systems results in substantial capital cost savings and reduction in total energy consumption and emissions.

In addition to the benefit of reducing operating and capital costs, a modified PDH plant or system of the present disclosure, in particular a plant or system configured to produce acrylonitrile, is also a greener process that generates lower carbon dioxide emissions than existing stand-alone plants or systems. The teachings of the present disclosure may be applied to the production of propene, ammonia, of acrylonitrile, of high purity hydrogen, urea, and of methanol, especially if combined with process carbon dioxide effluent from the PDH plant.

A hydrogen or dihydrogen recovery unit as disclosed herein is configured to separate out a stream of hydrogen from a stream of waste gas. This hydrogen stream is to be supplied from the hydrogen recovery unit in the propene production system of the modified PDH plant or system to another plant or system disposed downstream of the hydrogen recovery unit.

According to one embodiment, the modified PDH system or plant includes a cold box that is operatively disposed upstream of the hydrogen recovery unit or the hydrogen purification device. Alternatively, or in addition, the PDH plant may also include a CO₂ removal system that is operatively disposed upstream of the hydrogen recovery unit or the hydrogen purification device. A system configured in this manner is advantageous over prior art systems in that, for example, when linking the propene production system of the modified PDH system or plant with an acrylonitrile (AN) plant that is disposed downstream of the hydrogen recovery unit, acrylonitrile may be produced using only propane as feed stock.

In one aspect of an embodiment of the present disclosure, the modified PDH system or plant and its respective process includes a fuel system configured to burn waste gas to operate the modified PDH system. In this embodiment the hydrogen recovery unit or the hydrogen purification device is operatively disposed upstream of the fuel system. The hydrogen recovery unit is configured to recover hydrogen from the waste gas stream in the modified PDH plant or system, thereby diverting the hydrogen gas from the waste stream upstream of the fuel system so that it is not burned as fuel in the fuel system. With this configuration, various waste gasses present in the waste streams, primarily methane (CH₄), ethylene (C₂H₄), and ethane (C₂H₆), can be channeled directly to the fuel system.

In an aspect of one embodiment, the hydrogen purification device is disposed downstream of a CO₂ removal system or a de-ethanizer stripper system of the PDH plant, the hydrogen purification device being operatively disposed between a cold box and a fuel system of the PDH plant, the hydrogen recovery unit being operatively connected to an ammonia synthesis plant or system. This configuration of the PDH plant or system enables the recovery of hydrogen at an optimal location or stage of the PDH process in the modified PDH system. In this manner, PDH technology can be integrated or interconnected with ammonia plant technology in a very synergistic and efficient manner.

As mentioned previously, conventional ammonia production, which occurs in a stand-alone plant, accounts for approximately 80% of the total energy required for production of urea, acrylonitrile and other chemicals that require ammonia as a feed stock reactant. By utilizing the hydrogen in the modified PDH system as a secondary product, ammonia can be produced in a more efficient way, especially by adding a hydrogen purification step 7.1, in order to produce high purity hydrogen (99.999%). In particular for acrylonitrile production systems, that system interconnection may provide for more energy efficient and economically beneficial production methods.

In particular, it has been found that such an interconnection between a PDH plant and another plant, such as an ammonia plant, may also be realized in an economically feasible way when a standalone ammonia plant is not feasible, for example when the ammonia plant is intended for small scale production. Such a use of hydrogen is also favorable in that, in another stage, the newly produced ammonia may be provided from the ammonia synthesis loop to an acrylonitrile plant as a feedstock to produce acrylonitrile. In particular, this efficient and beneficial use of hydrogen significantly minimizes carbon dioxide emissions of ammonia plants generated in process as byproduct, or in the vent stack of steam methane reformers.

The present disclosure may provide for the following configuration. A PDH process is provided for acrylonitrile production and mainly for another plant like a polypropylene plant, wherein at least 17.5% of total propene (i.e. excess propene) may be routed to the polypropylene plant. Two outcomes are possible: The hydrogen recovery/purifier unit may be designed in a quite cost effective or technically straight forward manner, and/or excess ammonia can be produced in addition to ammonia required for acrylonitrile production by current ammoxidation (SOHIO) process.

According to one embodiment, the hydrogen recovery unit is connected to an ammonia synthesis loop that produces ammonia and directs a stream of ammonia to still another plant or system further downstream, especially to an acrylonitrile production plant. Alternatively or in addition, the hydrogen recovery unit may be operatively connected to an acrylonitrile production plant, especially for example by being used as feed stock to an ammonia synthesis loop to produce ammonia, which ammonia is then sent to the acrylonitrile production plant to produce acrylonitrile. An Air Separation Unit (ASU) provides high purity nitrogen to the synthesis gas compression unit, together with the hydrogen from the PDH process, which is needed for ammonia production in the ammonia synthesis loop. In particular, nitrogen that is needed to produce ammonia is provided to a synthesis gas compression unit upstream of the ammonia synthesis loop.

According to one embodiment, an at least one additional plant is an acrylonitrile plant that includes the ammonia synthesis loop as a part thereof, wherein both the hydrogen stream from the hydrogen recovery unit and propene (or propylene) are provided to the acrylonitrile plant as feed stock. Thereby, the modified PDH process of the present disclosure enables high efficiency synthesis of additional chemical end products downstream from the PDH process.

According to one embodiment, the PDH plant includes a C3 splitter system that is configured to distribute or route propene (C3H6) directly to the at least one additional plant (e.g. the acrylonitrile plant or system) downstream of the C3 splitter system. This embodiment allows for the supply of both propene and hydrogen or ammonia (if the hydrogen stream is supplied as the feed stock to an ammonia synthesis loop to produce ammonia) in an efficient manner.

A C3 splitter system may be understood as an assembly for separating a feed mixture stream of propane and propene/propylene into separate streams of high purity propane (C₃H₈) and propene/propylene (C₃H₆).

One advantage of a system as disclosed herein includes the efficient use of propene and hydrogen products from a PDH process, as feed stocks to further efficiently produce additional chemical products downstream from the PDH process in a single overall system. A modified PDH system as disclosed herein includes a propene production system configured to produce both propene and hydrogen from propane, by PDH. To do so, the modified PDH system includes a hydrogen recovery unit and a hydrogen purification unit that are configured to recover high purity hydrogen during the PDH process. The system as disclosed herein further includes a second plant or system connected to the PDH plant, for example an acrylonitrile plant, that uses the propylene/propene and hydrogen from the PDH plant as feed stock to efficiently produce additional chemicals, for example acrylonitrile. An excess stream of propylene/propene is also generated by the PDH plant, which can be further processed onsite or sold as an intermediate propylene/propene product.

According to one embodiment, the system of the present disclosure is configured to produce propene and hydrogen or ammonia in a PDH plant/system, wherein a second plant may be configured to produce ammonia, or acrylonitrile, or acrylic acid. In this manner, considerable synergetic effects may be achieved for the production of one or more of the chemicals disclosed herein, as described above. Such a system may also be configured to produce propene and ammonia simultaneously based on a minimum molar ratio of propene:ammonia of 1:0.9897, by utilizing the hydrogen byproduct from said PDH process for ammonia synthesis as described above. Such a method allows for improving the propane utilization efficiency.

According to one embodiment, propene is provided both as partial propene stream to an acrylonitrile plant and as excess propene stream to any further plant or as end product, the excess propene stream being at least 1% of total propene output from the PDH process, and may be at least 16%, or at least 16.5%, or at least 17.5% depending on the excess ammonia required for the propene ammoxidation reaction. This allows for high efficiency use of propane and/or acrylonitrile production. Excess propene may be considered the molar ratio of the amount of propene not participating in acrylonitrile production to the total propene produced by the PDH process.

According to one embodiment, hydrogen recovered from the PDH process is mixed with nitrogen, especially nitrogen from an air separation unit ASU, especially in a molar ratio of 2.9:1 or 3:1. Thereby, ammonia may be produced with high yield.

According to one embodiment, a waste stream downstream of the hydrogen recovery unit is conveyed to a fuel system of the PDH plant/system, together with make-up fuel, where the waste stream and make-up fuel are consumed/burned in the fuel system to generate energy to run the PDH plant. Using the waste stream as feed to the fuel system minimizes the amount of make-up fuel that must be supplied to the fuel system for it to run at full capacity. The waste stream is comprised of any unneeded byproduct hydrocarbon chemicals resulting from the PDH process, including for example methane (CH4), ethylene (C2H4), and/or ethane (C2H6) separated out by the hydrogen recovery unit. Thereby, efficiency of PDH process is maintained at a high level.

According to one embodiment, up to 99.95% of hydrogen is recovered, especially downstream of the cold box system in the modified PDH system. This recovering rate provides for considerable synergies. According to one embodiment, at least 70% or 80% or 90% or 95% of hydrogen is recovered from the hydrogen rich gas product from the modified PDH process.

According to one embodiment, the recovered hydrogen is used to generate an ammonia stream as feed stock to an acrylonitrile production plant operatively disposed downstream of the ammonia production system. Alternatively or in addition, a method may comprise recovering hydrogen in order to provide ammonia in conjunction with propene to the second plant, especially to an acrylonitrile plant, wherein exclusively propane is used as feedstock.

According to one embodiment, recovering hydrogen is carried out for production of acrylonitrile, and/or for production of acrylic acid, and/or for methanol synthesis, and/or for polymerization reactions.

Such a use of hydrogen may provide for elimination of the energy-intensive front end of conventional stand-alone ammonia plants, resulting in significant operating cost savings relative to standalone ammonia plants.

### BRIEF DESCRIPTION OF THE DRAWINGS

The present invention is described in detail below with reference to the attached drawing figures, wherein:

FIG. 1a is a flow chart depicting a schematic high-level embodiment of a conventional plant for producing acrylonitrile by the process of propene (propylene) ammoxidation;

FIG. 1b is a flow chart depicting a schematic high-level embodiment of a conventional plant for producing acrylonitrile by the process of propane ammoxidation;

FIG. 2 is a flow chart depicting a schematic embodiment of a conventional propane dehydrogenation (PDH) plant or system;

FIG. 3 is a flow chart depicting a schematic embodiment of a modified PDH plant or system of the present disclosure for producing both acrylonitrile and excess propene;

FIG. 4a is a simplified flow chart depicting a schematic embodiment of a modified PDH plant or system of the present disclosure for producing both acrylonitrile and excess propene.

FIG. 4b is a simplified flow chart depicting a schematic embodiment of a modified PDH plant or system of the present disclosure for producing both propene and hydrogen.

FIG. 4c is a simplified flow chart depicting a schematic embodiment of a modified PDH plant or system of the present disclosure for producing both ammonia and propene.

FIG. 4d is a simplified flow chart depicting a schematic embodiment of a modified PDH plant or system of the present disclosure for producing both urea and propene.

FIG. 4e is a simplified flow chart depicting a schematic embodiment of a modified PDH plant or system of the present disclosure for producing both methanol and propene.

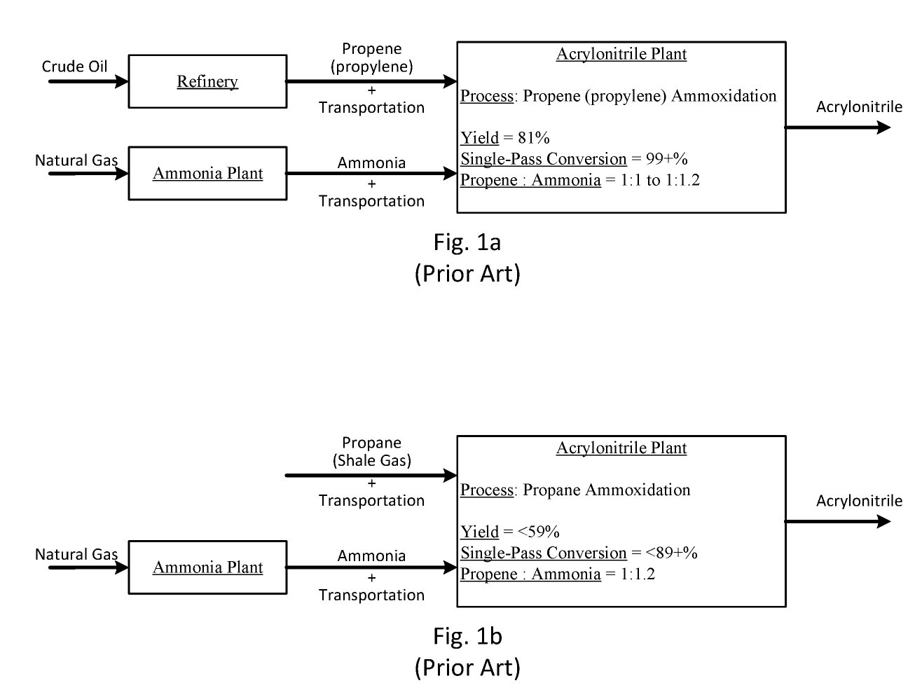

### DETAILED DESCRIPTION

Various embodiments now will be described more fully hereinafter with reference to the accompanying drawings, which form a part hereof, and which show, by way of illustration, specific embodiments. However, the present disclosure may be embodied in many different forms and should not be construed as limited to the embodiments set forth herein. Rather, these embodiments are provided so that this disclosure will be thorough and complete, and will fully convey the scope of the present disclosure to those skilled in the art. The following detailed description is not to be taken in a limiting sense.

Throughout the specification and claims, the following terms take the meanings explicitly associated herein, unless the context clearly dictates otherwise. The phrase “in one embodiment” does not necessarily refer to the same embodiment, although it may. Furthermore, the phrase “in another embodiment” does not necessarily refer to a different embodiment, although it may. Thus, as described below, various embodiments may be readily combined without departing from the scope or spirit of the present disclosure.

In addition, as used herein, the term “or” is an inclusive “or” operator, and is equivalent to the term “and/or,” unless the context clearly dictates otherwise. The term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

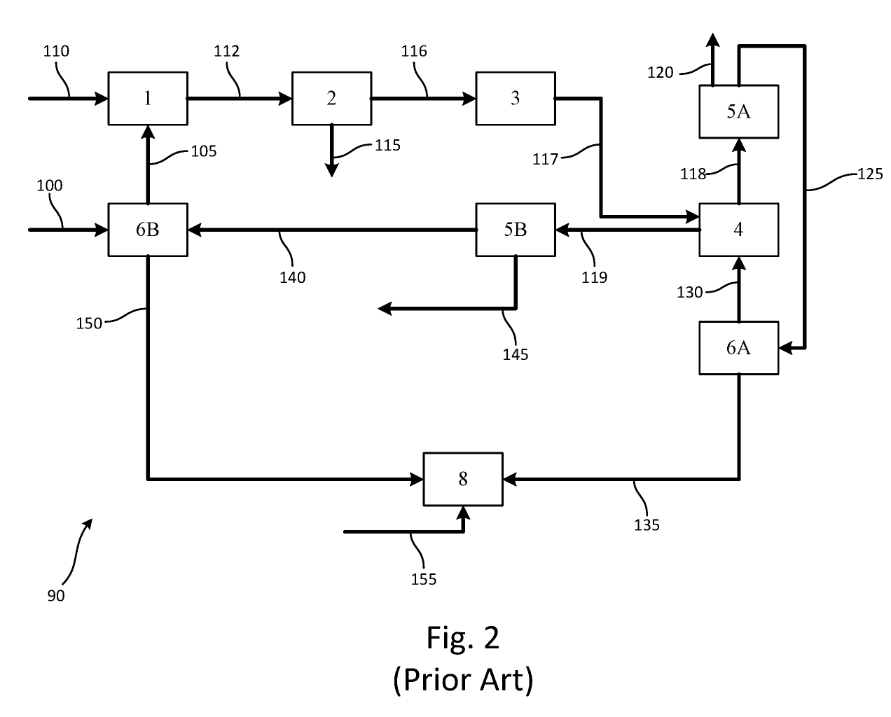

Referring to FIG. 2, a schematic diagram of a conventional PDH plant or system 90 for producing propene (C₃H₆), including various components or subsystems thereof, depicting a conventional PDH process, is shown. Production of propene (C₃H₆), which is also commonly known as propylene, in a conventional PDH system 90 is based on the following chemical reaction, whereby propane (C₃H₈) 100 is reacted to produce propene (C₃H₆) and hydrogen (H₂):

C₃H₈→C₃H₆+H₂

This reaction is highly endothermic (124 kJ/mol) and high reaction temperatures are necessary to achieve high propane conversion. In one embodiment, a propane dehydrogenation may be performed, for example, over a Pt—Sn/alumina catalyst in one or more fixed bed PDH reformer(s) under nearly atmospheric pressure and temperatures of 580-650° C. Side reactions also taking place in the reformer may include the following:

C₃H₈→CH₄+C₂H₄

In this reaction, propane (C₃H₈) is reacted to produce methane (CH₄) and ethylene (C₂H₄).

C₂H₄+H₂→C₂H₆

In this reaction, ethylene (C₂H₄) reacts with hydrogen (H₂) to produce ethane (CH₆).

C₃H₈+H₂→CH₄+C₂H₆

In this reaction, propane (C₃H₈) reacts with hydrogen (H₂) to produce methane (CH₄) and ethane (C₂H₆).

C₃H₈+6H₂O→3CO₂+10H₂

In this reaction, propane (C₃H₈) is reacted with steam (H₂O) to produce carbon dioxide (CO₂) and hydrogen (H₂).

As shown in FIG. 2, a feed stream of make-up propane (C₃H₈) 100 is treated in a depropanizer column 6B to remove any hydrocarbons heavier than propane that may be present in the feed stream. The hydrocarbons that are heavier than propane exit the depropanizer column 6B in a waste stream 150, while the purified stream of propane 105 is then mixed with steam 110 and preheated to the inlet temperature of a PDH reformer or reactor 1. A reformer outlet stream of process gas 112 exits the reformer 1 and contains primarily propane (C₃H₈), hydrogen (H₂), propene (C₃H₆), methane (CH₄), ethane (C₂H₆), ethylene (C₂H₄), carbon dioxide (CO₂), and steam (H₂O), and has a temperature of about between 580-600° C. It is understood that, in addition to the above exemplary listed chemicals, other small amounts of additional chemicals may be present in the process gas stream. All chemical reactions occur within the PDH reformer 1, and all subsequent process steps that occur downstream are merely separation process steps to separate out various of the aforementioned chemical constituents present in the process gas stream 112. Next the process gas 112 is sent from the reformer 1 to a waste heat recovery system 2 where steam is first separated out as steam condensate 115 from the process gas stream 112.

The cooled process gas 116 leaves the waste heat recovery system 2 and enters a raw gas compression unit 3 where it is compressed to around 32 bar. This compression is necessary for the subsequent distillation and CO₂ removal systems downstream. From the raw gas compression unit 3, the compressed and cooled process gas 117 is then sent to a de-ethanizer stripper system 4. In the de-ethanizer stripper system 4, a stream of process gas containing any chemical component that is lighter than propene (C₃H₆), including carbon dioxide, 118 will be separated out from the compressed and cooled process gas 117 and sent to a CO₂ removal unit 5A. And a stream of process gas containing propene (or propylene) (C₃H₆) and any chemical component heavier than propene 119, which includes primarily propane (C₃H₈), will be sent to the C3 splitter system 5B. In the CO₂ removal unit 5A, carbon dioxide (CO₂) 120 is separated out from the process gas 118. The remaining components in the stream of process gas 125 are hydrocarbons and hydrogen together with small amounts of propane and propene (or propylene). This stream of process gas 125 is then sent from the CO2 removal unit 5A to the cold box 6A to recover any fugitive propane and propene remaining therein.

A cold box 6A may be understood as an assembly for separating chemical components of fluids or mixtures by partial condensation. A cold box 6A may comprise one or more of a heat exchanger, a boiler, a distillation column, an expander or expansion brake turbine, or other separation technologies. In some embodiments, an expander may be a component of the cold box 6A. In other embodiments, the cold box 6A itself (excluding any expander) can be part of a rectification column, acting as a condenser where operated as low as −186° C. Avoiding decompression of a hydrogen rich stream and thereafter having to recompress the stream (especially for ammonia synthesis) may serve to prevent the wasting of energy.

The cold box 6A partially condenses the process gas 125 into a propene (or propylene) and propane rich process gas stream 130, and also separates out a light hydrocarbon (including hydrogen) stream 135. The propene (or propylene) and propane rich process gas stream 130 is sent from cold box 6A the back to the de-ethanizer stripper system 4 for additional separation as previously discussed. Referring now back to the C3 splitter system 5B, the feed stream of process gas 119 is separated or distilled into a high purity propane stream 140 (C₃H₈) and a high purity propene (or propylene) stream 145 (C₃H₆). The high purity propane stream 140 is recycled back to the depropanizer system 6B and joins the make-up propane stream 100, where unreacted high purity propane from the high purity propane stream 140 participates in the reaction as discussed above. The propene (or propylene) stream 145 that is separated out in the C3 splitter system 5B is an intermediate product that may be sold or further used in other chemical processes.

Referring again the cold box 6A, the light hydrocarbon stream 135 (including hydrogen) is sent to a fuel system 8, together with the waste stream of heavier hydrocarbons 150 that was separated out from the make-up propane 100 in the depropanizer system 6B and a stream of make-up fuel 155, where they are all consumed or burned to generate the energy necessary to operate the conventional PDH plant or system 90. Using the light hydrocarbon stream 135 and the waste stream of heavier hydrocarbons 150 from the depropanizer 6B as feed for the fuel system 8 minimizes the amount of make-up fuel 155 (e.g. natural gas) consumption required by the fuel system 8 for it to run at full capacity to accommodate the heat energy requirements of the PDH system or plant 90. The light hydrocarbon stream 135 is comprised of any unneeded byproduct hydrocarbon chemicals resulting from the PDH process, including for example methane (CH4), ethylene (C2H4), ethane (C2H6), and hydrogen (H₂) that were separated out by the cold box 6A.

As discussed in the background section, one drawback of conventional PDH plants or systems is that it is only able to produce one end product (i.e. propene) using one feed stock (i.e. propane). In order to produce additional products (e.g. acrylonitrile) from the propene end product, additional stand-alone plants (i.e. ammonia plant), built at a considerable upfront capital cost and operational expense, are needed to produce other intermediary products and or a final end product. In the case of acrylonitrile production for example, propene produced in a stand-alone conventional PDH plant 90 must be transported (typically by semi-truck which incurs additional expense), together with ammonia produced in a separate stand-alone ammonia production plant (again by semi-truck at an additional expense) to the acrylonitrile plant, where they are both used together as feed stock to produce acrylonitrile.

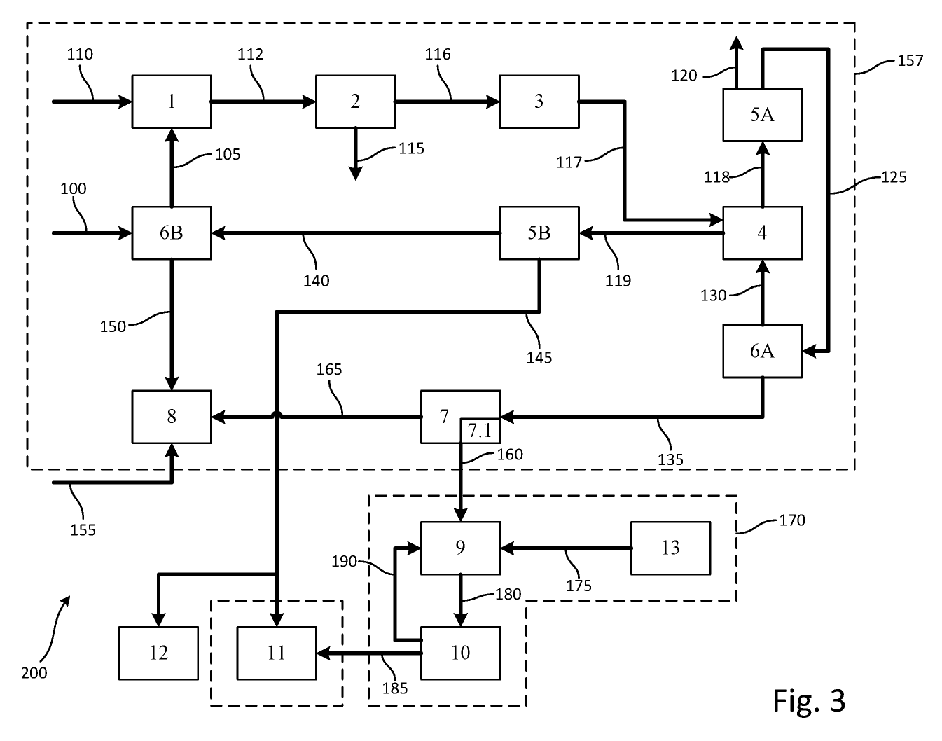

Referring now to FIG. 3, an embodiment of a modified PDH plant or system 200 is shown by which propane is used as the feedstock for the production of both excess propene and acrylonitrile. However, as will be described in further detail below, it will be apparent that the concept disclosed herein is flexible such that alternate embodiments of the modified PDH system of the present disclosure are able to produce (1) propene and hydrogen, or (2) propene and ammonia, or (3) propene and urea, or (4) propene and methanol, or other combinations thereof.

As discussed previously, conventional PDH plants or systems produce only propene as the sole product with all remaining byproducts (including hydrogen) being used as fuel to run the PDH plant or system. The modified PDH plant or system 200 of the present disclosure not only produces propene product from the PDH process, but also either produces hydrogen as an end product, or uses the hydrogen byproduct produced in the PDH process as one of the two required feed stocks for one or more additional downstream subsystem or process within the modified PDH process, such as for example, an acrylonitrile production system 11, an ammonia production system or plant 170, a urea production system (not shown), or a methanol production system (not shown). In this manner, the modified PDH system 200 is able to simultaneously produce propene from the PDH process 200, as well as one or more of ammonia, acrylonitrile, urea, and/or methanol. Thus, this modified PDH plant or system 200 can simultaneously produce at least two valuable products from the single modified PDH system or plant and process, all while using propane as the sole feed stock to produce all of the aforementioned products.

Referring still to FIG. 3, in the depicted embodiment of the modified PDH plant or system, the feed stream of make-up propane 100 is processed in a propene production system 157 of the modified PDH plant or system 200 primarily in a similar manner, through the depropanizer system 6B, the PDH reformer 1, the waste heat recovery system 2, the raw gas compression unit 3, the de-ethanizer stripper system 4, the CO₂ removal system 5A, the C3 splitter 5B, and the cold box 6A, as is done in the conventional PDH plant or system 90 shown in FIG. 2. However, the modified PDH plant or system 200 shown in FIG. 3 diverts from the design of the conventional PDH plant or system is several key respects, including, among other aspects, adding several components or systems that are not present in a conventional PDH system or plant that are configured to take advantage of synergies between processes and systems, and more efficiently use the hydrogen and propene present in gas streams at various stages in the modified PDH process 200.

Referring further to FIG. 3, the depicted embodiment of the modified PDH process 200 of the present disclosure includes a propene production system 157 that contains primarily the same systems/components present in the conventional PDH system, but also now includes a hydrogen recovery unit 7 configured to recover hydrogen (H₂) from at least one of a hydrogen containing process gas stream, or from one of the byproduct or waste streams that contain hydrogen, in the propene production system 157.

In one embodiment, the separated/recovered stream of hydrogen recovered from the hydrogen recovery unit 7 has an ultra-high purity, for example greater than 99.9%, or greater than 99.99%, or greater than 99.999%.

The hydrogen recovery unit 7 may have various configurations (which may also include one or more hydrogen purification devices contained therein) and operate based on various exemplary technologies, several of which are discussed below and/or referenced in the table below. In the embodiment depicted in FIG. 3, the hydrogen recovery unit 7 includes a hydrogen purification device 7.1 configured to purify hydrogen recovered by the hydrogen recovery unit 7. According to one embodiment, the hydrogen recovery unit, including a hydrogen purification device, utilizes membrane technology (e.g. polymer membrane, metallic membrane, etc.) for the recovery and/or purification of hydrogen from a feed stream, and is operated at pressures up to 138 bar, within a temperature range of 250 to 450° C. In still other alternate embodiments, as shown in the table below, a hydrogen recovery unit may utilize a membrane technology (as disclosed above), a Pressure Swing Adsorption (PSA) technology, or a cryogenic technology, either alone or in combination with another technology disclosed herein.

As discussed above, several known exemplary hydrogen recovery and/or purification technologies and their associated operating conditions and ranges are listed below for reference:

| | | Operating | Operating | Feed H₂ | Purity of | Recovery of |
|---|---|---|---|---|---|---|
| | | Pressure | Temperature | molar | hydrogen | H₂ (%) from |
| Method | Example | (bar) | (° C.) | concentration | product % | feed stream |
| partial | cryogenic fluids, | up to and | down to | more than | up to | up to |
| condensation | like liquid nitrogen | more than | −186 | 30% | 99.54 | 99.95% |
| | | 120 | | | | |
| PSA (only | adsorbents | 20-150 | ambient | more than | up to | more than |
| hydrocarbons) | | | | 60% | 99.99 | 70% |
| PSA | adsorbents | 10-40 | ambient | 50-80% | up to | 70-96% |
| (hydrocarbons | | | | | 99.999 | |
| with CO and CO₂) | | | | | | |
| polymer | polyamides or | 20-200 | 0-100 | 70-90% | up to | more than |
| membranes | polysulfone | | | | 99.9+% | 85% |
| metallic | palladium | up to | 250 to more | more than | up to | up to |
| membranes | silver alloy | 138 | than 400 | 90% | 99.999+% | 99% |
| metal | lanthanum- | less than | more than | less than | 99% | more than |
| hydrides | nickel based alloy | 40 | 30 | 60% | | 90% |

Using the technologies discussed above, the hydrogen recovery unit may be configured to output ultra-high purity hydrogen having a pressure ranging from atmospheric pressure up to 200 bar, and a temperature ranging from −190° C. up to 400° C., and having a purity of up to 99.999%.

As between the previously disclosed technologies shown in the table above, it has been found that utilizing cryogenic separation technologies may be quite favorable in conjunction with the recovery and purification of hydrogen in a modified PDH system, since cryogenic separation may provide for a high hydrogen recovery rate (99+%) with hardly any noticeable impurities included (e.g. in the range of only 0.3%). The hydrogen stream from the hydrogen recovery unit may then be supplied to a PSA process or a membrane process, which can recover at least 96% of hydrogen from the waste gas feed stream at the aforementioned ultra-purity levels.

According to one embodiment, the hydrogen recovery unit includes a hydrogen purification device configured to purify hydrogen product produced by a PDH plant. The purity of such purified hydrogen is, for example, higher than 99.999% (ultra-high purity hydrogen). A hydrogen product having such a purity level allows for several procedural advantages, especially when used as a feed gas for ammonia production or acrylonitrile production. In particular, high purity hydrogen can be utilized as feedstock for additional processes downstream of the hydrogen recovery unit, for example ammonia production systems, acrylonitrile production systems, hydrotreating systems, urea production systems, and/or methanol systems where additional chemical products may be produced. Hydrogen at such purity levels is beneficial in that it can minimize hydrogen losses, especially in methanol or ammonia synthesis loops. A modified PDH plant that includes a hydrogen recovery unit as discussed above can provide a very competitive and economic system for the simultaneous production of high purity hydrogen and propene.

The hydrogen recovery unit (including the hydrogen purification device) may be configured based on the specific recovery method or technology that will be utilized, for example for a membrane system providing for a maximum recovery of up to 99% and a maximum purity of up to 99.99999%, or for pressure swing adsorption (PSA) providing for a recovery of 70% to at least 90% and a maximum purity of at least 99%, or for a cryogenic process providing for a maximum recovery of up to 99.95% and a purity of at least 99.9%.

In the embodiment shown in FIG. 3, the hydrogen recovery unit 7 is operatively disposed downstream of the cold box 6A, between the cold box 6A and the fuel system 8. At this location, the light hydrocarbon stream 135 (including hydrogen) from the cold box 6A enters the hydrogen recovery unit 7 where it is separated into a stream of purified hydrogen 160, and a separate stream of the remaining light hydrocarbons 165, such as for example methane (CH₄), ethane (C₂H₆), and ethylene (C₂H₄). In one embodiment, the stream of purified hydrogen 160 is sent to a simplified ammonia production plant or system 170, while the stream of light hydrocarbons is sent to the fuel system 8 to be burned/consumed as described above in relation to FIG. 2 and the conventional PDH plant or system.

It has been found that this location between the cold box 6A and the fuel system 8 is the optimal location at which to insert the hydrogen recovery unit 7 in order to achieve the most efficient and complete separation of hydrogen from the gas streams flowing through the various stages and systems in the PDH process. Having the hydrogen recovery unit 7 disposed downstream of the cold box 6A enables the recovery of as much as 99.95% of the hydrogen contained in the stream of light hydrocarbons and hydrogen 135 coming from the cold box 6A, with this recovered hydrogen having an ultra-high purity of 99.999%. In order to recover such a high percentage of hydrogen at such an ultra-high purity, the hydrogen recovery unit 7 may utilize one or more technologies, such as membrane separation technologies, especially those utilizing Pd/Ag membranes, cryogenic separation technologies, physical adsorption, and/or various combinations thereof.

However, while the above described embodiment shown in FIG. 3 discloses the hydrogen recovery unit 7 being disposed between the cold box 6A and the fuel system 8, this disclosure should not be read to limit the location in the modified PDH system 200 at which the hydrogen recovery unit 7 may be disposed. Rather, in alternate embodiments, the hydrogen recovery unit 7 may be operatively disposed in the modified PDH system at alternate locations without departing from the scope of the present disclosure. For example, in an alternate embodiment the hydrogen recovery unit 7 may be operatively disposed at any other position downstream of the PDH reformer 1. In still alternate embodiments, the hydrogen recovery unit 7 may be disposed between the PDH reformer 1 and the waste heat recovery unit 2, or between the waste heat recovery unit 2 and the raw gas compression unit 3, or between the raw gas compression unit 3 and the de-ethanizer stripper system 4, or between the de-ethanizer stripper system 4 and the CO₂ removal system 5A, or between the CO₂ removal system 5A and the cold box 6A, or between the cold box 6A, and the de-ethanizer stripper system 4, or between the de-ethanizer stripper system 4 and the C3 splitter system 5B, or between the C3 splitter system 5B and the depropanizer system 6B.

Furthermore, while the embodiment shown in FIG. 3 depicts the hydrogen purification device 7.1 being a part of the hydrogen recovery unit 7, in alternate embodiments, the hydrogen purification device 7.1 may be a separate system or device from, and/or external to, the hydrogen recovery device 7 and may be disposed anywhere between the CO₂ removal unit 5A and a fuel system 8 of the PDH plant.

Continuing on with reference to FIG. 3, an embodiment of the modified PDH plant or system 200 and process also includes a simplified ammonia production plant or system 170, as compared to conventional stand-alone ammonia production plants which will be discussed in further detail below. The simplified ammonia production system 170 is operatively coupled to the hydrogen recovery unit 7 in the propene production system 157. The ammonia production system 170 is configured to use the hydrogen stream 160 from the hydrogen recovery unit 7 as a feed for the production of ammonia (NH₃). The simplified ammonia production plant or system 170 is simplified as compared to conventional ammonia production plants in that it comprises merely a synthesis gas (“syn. gas”) compression unit 9, an ammonia synthesis loop 10, and an air separation unit (“ASU”) 13. The ASU 13 separates air into its various chemical components, which includes a stream of high purity nitrogen 175 (anything at or greater than 99.99% pure N₂) that, together with hydrogen (H₂), is needed as feed stock to produce ammonia (NH₃). The stream of purified hydrogen 160 from the hydrogen recovery unit 7 is mixed with the stream of high purity nitrogen 175, for example in a molar ration of hydrogen: nitrogen of 2.9:1, or 3:1, or any other functional ratio, and sent to the syn. gas compression unit 9 as make-up gas where it is compressed. This make-up gas contains no inert content, as would otherwise be present in a conventional ammonia synthesis production plant. The compressed stream of hydrogen and nitrogen 180 is then sent downstream to an ammonia synthesis loop 10 where it is reacted to form ammonia (NH₃). A stream of ammonia 185 exits the ammonia synthesis loop 10 as an end, or intermediate, product to be used in other processes (e.g. as feed for an acrylonitrile plant 11, or a urea plant [not shown], or nitric acid). In addition, a separate stream of unreacted hydrogen and nitrogen 190 leaves the ammonia synthesis loop 10 and is recycled back to the syn. gas compression unit 9 where it joins the incoming hydrogen stream 160 and nitrogen stream 175 to be recompressed and sent back to the ammonia synthesis loop 10 to be reacted to generate ammonia as previously disclosed. In one embodiment, the modified PDH system and process may provide for a minimum molar ratio of propene:hydrogen:ammonia of 1:1.484:0.989. In particular, in a standalone PDH process, for every 1 mol of propene product, up to 0.9897 mol of ammonia may be produced.

As stated above, the simplified ammonia production plant or system 170 is simplified as compared to conventional ammonia production plants. Conventional ammonia production plants or systems typically utilize natural gas as both a fuel to be burned to run the conventional ammonia plant, and as a feed stock to create the hydrogen that is required to produce ammonia in the ammonia synthesis loop of the ammonia production plant. And the process of turning natural gas into hydrogen in the front-end of a conventional ammonia production plant, using for example coal gasification, steam methane reforming, or other conventional technologies located upstream of an ammonia synthesis loop, is not only very energy-intensive and expensive to run, but this front-end process also requires a significant capital investment in the expensive equipment needed just to turn the natural gas into hydrogen. For example, such a front end of a conventional ammonia production plant typically takes the natural gas feed stock and processes it in each of a purification and saturation device, a steam reformer, a CO conversion system, a desaturator system, a CO₂ removal system, and a methanation system, all of which requires significant capital investment to purchase and install, and large amounts of energy to operate, which means routinely high operating costs.

In contrast, production of ammonia with the modified PDH plant or system 200 and process of the present disclosure eliminates this front end energy-intensive process, with its high operating cost, expensive front end equipment, and large capital expense, because the propene production system 157 of the modified PDH system 200 is able to generate all of the purified hydrogen feed that is needed by the simplified ammonia synthesis loop 10 in order to produce ammonia. Thus, to produce ammonia in addition to propene 145, the modified PDH plant 200 need only include this cheaper, greener/cleaner, and more energy efficient simplified ammonia production plant or system 170, without the front-end systems present in conventional ammonia plants. This at least ensures capital and operating cost savings, primarily due to elimination of a substantial part of the conventional ammonia plant.

In addition to the reduced capital and operation costs, another additional benefit of the modified PDH system or plant 200 of the present disclosure is that the simplified ammonia production plant or system 170 included in some embodiments, like that shown in FIG. 3, is significantly more energy efficient than a conventional ammonia production plant. Additionally, utilizing the simplified ammonia production plant or system 170 as part of the modified PDH system 200 eliminates the carbon dioxide (CO₂) emissions that are usually associated with the front end process of a conventional stand-alone ammonia plant, whether these emissions are a by-product CO₂ from the front-end process itself, or CO₂ emissions generated from burning fuel. Thus, embodiments of the modified PDH system 200 that include the simplified ammonia production system 170 are significantly greener than conventional ammonia production systems.

Referring further to FIG. 3, in one embodiment, the modified PDH plant or system 200 may further include an acrylonitrile plant or system 11 that is operatively coupled thereto, downstream of the C3 splitter system 5B in the propene production system 157, and downstream of the ammonia synthesis loop 10 of the simplified ammonia production plant 170. The acrylonitrile plant or system 11 operates based on the propene (propylene) ammoxidation process and is configured work with the propene production system 157 and the ammonia production system 170 to produce acrylonitrile as another end product in the modified PDH system 200. In such an embodiment of the system 200, the high purity propene (C₃H₆) stream 145 is sent from the C3 splitter system 5B, and the ammonia (NH₃) stream 185 is sent from the ammonia synthesis loop 10 of the simplified ammonia production plant 170, to be used as feed stock to the acrylonitrile plant 11 where acrylonitrile is produced by the propene ammoxidation process.

Any excess propene 12 produced in the propene production system 157 that is not needed for the production of acrylonitrile may be sold off, or further used as an intermediary or feed stock to produce other products requiring propene, such as for example acrylic acid, polypropylene, propene oxide, etc. For example, under certain operating parameters, at least 16.5% of the total mass of propene 12 produced by the modified PDH system 200 may be excess propene 12. In the acrylonitrile chemical reaction, a molar ratio of propene:ammonia of 1:1 may be considered. The actual ratio of propene:ammonia is between about 1:1 to 1:1.2 in most technologies of propene ammoxidation to minimize complete oxidation of propene. When an acrylonitrile plant requires a molar ratio of propene:ammonia (SOHIO ammoxidation process) of 1:1.2, then the excess propene could be at least 17.58%, for a PDH process built for acrylonitrile production. In case the acrylonitrile plant may require only a molar ratio of propene:ammonia of 1:1, then the excess propene could be at least 1%, for a PDH process built for acrylonitrile production.

However, it should be understood that the amount of excess propene that may be produced with acrylonitrile production is not limited to these exemplary percentages, and that alternate amounts may be produced depending on the operating parameters of the modified PDH system 200. As an another alternative, all of the propene produced by the modified PDH system 200 (including any that would otherwise be considered excess propene 12) may be used to produce acrylonitrile, and any shortage in the amount of ammonia required to react with the excess propene 12 could be made up by purchasing additional ammonia, so that the system produces only acrylonitrile as an end product. Including the acrylonitrile system 11 as part of the modified PDH process 200 enables the use of propane 100 as the sole feed stock for the production of any one or more of purified propene, hydrogen, ammonia from the hydrogen, and/or acrylonitrile from the propene and ammonia, and otherwise provides the most cost competitive, green, and energy efficient system and method for the production of acrylonitrile that exists to date.

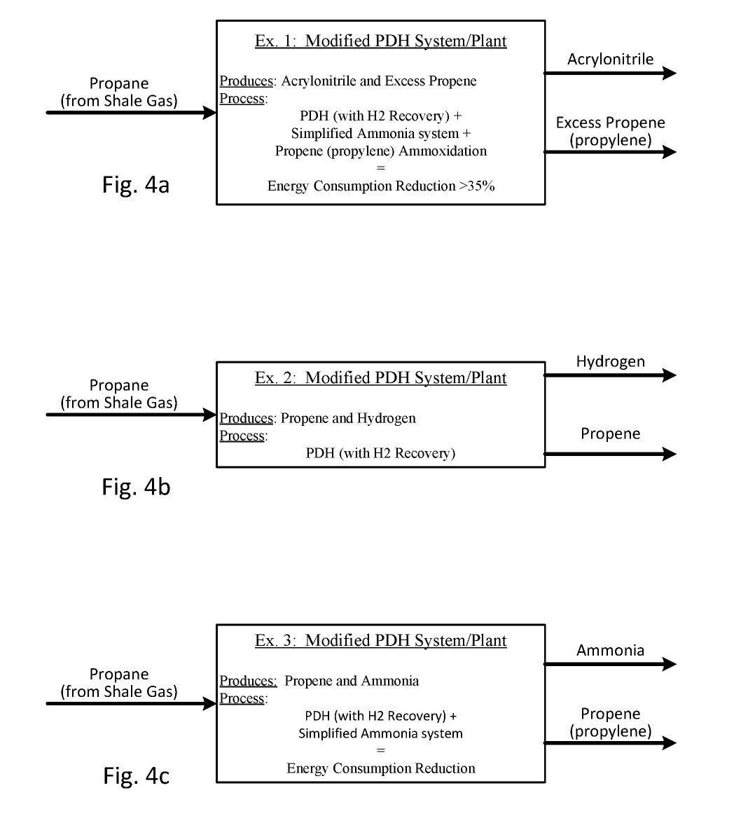

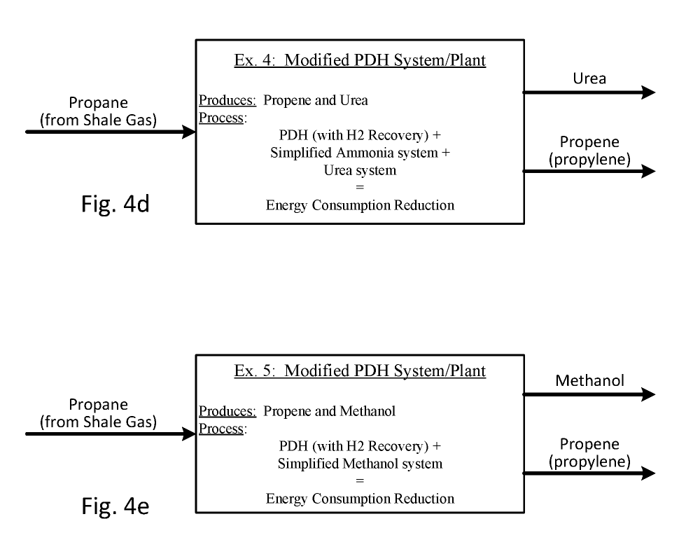

As discussed above, an embodiment of the modified PDH process 200 shown in FIG. 3 is configured primarily for the production of acrylonitrile and excess propene end products. This embodiment of the modified PDH process 200 includes the propene production system 157, the ammonia production system 170, and the acrylonitrile production system 11. This embodiment is also shown on a higher lever in FIG. 4a, with one of the benefits of such an embodiment being a net energy consumption reduction of more than 35% for the entire process 200 as opposed to producing acrylonitrile by conventional methods using stand-alone plants or systems, and transporting various chemical end products and feed stocks.

However, referring to FIG. 4b, another alternate embodiment of the modified PDH system 200 comprises solely the propene production system 157 that includes the hydrogen recovery unit 7 disposed between the cold box 6A and the fuel system 8. This embodiment of the system 200 is configured to produce ultra-high purity hydrogen and propene as the two sole end products. In this embodiment, the ammonia production system (170 in FIG. 3), and/or the acrylonitrile production system (11 in FIG. 3) respectively either do not form a part of the system 200 or are not being operated, or a combination thereof.

Referring to FIG. 4c, still another alternate embodiment of the modified PDH system 200 comprises the propene production system 157 that includes the hydrogen recovery unit 7, and the ammonia production system 170. This embodiment of the system 200 is configured to recover both propene and ammonia as the end products. And in some embodiments may be configured to also divert a portion of the hydrogen as an end product. In this embodiment, the acrylonitrile production system 11 either does not form a part of the system 200, or is at least not being operated to produce acrylonitrile.

Referring to FIG. 4d, still another alternate embodiment of the modified PDH system 200 comprises the propene production system 157 that includes the hydrogen recovery unit 7, and the ammonia production system 170. In this embodiment, a urea system (not shown in any drawing) is also operatively coupled downstream of the ammonia system 170 shown in FIG. 3. This urea system may be swapped-in in place of the acrylonitrile system 11 in FIG. 3, or may be connected in parallel to it such that the stream of ammonia may be diverted to either the acrylonitrile system or the urea system. Regardless of the specific configuration, this embodiment of the system 200 is configured to recover at least propene and urea as the end products. And in some embodiments this modified PDH system 200 may be configured to also divert a portion of the hydrogen or ammonia as an end product. To produce the urea in the present embodiment, the fuel burned in the fuel system 8 produces waste combustion gas containing CO₂. This CO₂, or any other emissions containing CO₂ that are generated by the PDH system, is sent to a CO₂ removal system to separate out the CO₂. The separated CO₂, and the ammonia from the ammonia production system 170, are both sent to the urea synthesis system to produce urea as an end product. In this embodiment, the acrylonitrile production system 11 either does not form a part of the system 200, or is at least not being operated to produce acrylonitrile.

Referring to FIG. 4e, still another alternate embodiment of the modified PDH system 200 comprises the propene production system 157 that includes the hydrogen recovery unit 7. In this embodiment, a methanol system (not shown in any drawing) that includes a methanol synthesis loop is also operatively coupled downstream of the propene production system (157 shown in FIG. 3). The schematic set up of the methanol system would be similar to that shown in FIG. 3 for the ammonia system 170. This methanol system may be swapped-in in place of the ammonia system 170 in FIG. 3, or may be connected in parallel to it such that the stream of hydrogen from the propene production system 157 may be diverted to either the ammonia system or the methanol system. Regardless of the specific configuration, this embodiment of the system 200 is configured to recover at least propene and methanol as the end products. And in some embodiments this modified PDH system 200 may be configured to also divert a portion of the hydrogen as an end product, or a portion of the hydrogen to an ammonia system 170 in parallel to also produce ammonia as an end product, or any other product that uses ammonia downstream as feed stock. To produce the methanol in the present embodiment, the fuel burned in the fuel system 8 produces waste combustion gas containing CO₂. This CO₂, or any other emissions containing CO₂ that are generated by the PDH system, is sent to a CO₂ removal system to separate out the CO₂. The separated CO₂, is sent to a syngas compression unit. The compressed CO₂ and the hydrogen (160 in FIG. 3) from the propene production system 157 are both sent to a methanol synthesis loop which thereafter passes through a methanol separation/purification system to produce methanol as an end product. In this embodiment, the ammonia system 170 and the acrylonitrile production system 11 either do not form a part of the system 200, or are at least not being operated to produce either ammonia or acrylonitrile.

Furthermore, still additional benefits to the modified PDH system 200 of the present disclosure exist as compared to conventional PDH plants or systems and processes. For example, in embodiments of the modified PDH system 200 that include at least the propene production system 157 and the simplified ammonia production system 170, as well as embodiments that also include an acrylonitrile system 11, there exists a favorable and unanticipated process and system integration synergy between the different systems and processes (i.e. between the propene production system 157 and the ammonia production system 170, and/or the acrylonitrile plant 11). For example, in embodiments of the modified PDH system 200 that include the acrylonitrile plant 11, both of the ammonia production system 170 and the acrylonitrile production system 11 and their associated processes are net exporters of steam, while the propene production system 157 and its process is a net importer of steam. The propene production system 157 uses steam that is fed to the PDH reformer 1 to decrease the partial pressure of the propane needed for the reaction to occur in the PDH reformer 1. Also, steam is used to drive various steam turbines included in various of the subsystems in the modified PDH system 200, such as for example in the raw gas compression unit 3, in the syn. gas compressor 9, in an ammonia refrigeration compressor (not shown) that forms a part of the ammonia synthesis loop 10, and in still various others. By using the excess steam generated by the ammonia production system 170 and/or the acrylonitrile production system 11 in the manner disclosed above, the size of a boiler required by the modified PDH system 200 may be reduced or minimized, as compared to conventional PDH systems, further resulting in higher efficiency and lower CO2 emissions.

Still other advantages and synergies exist within the modified PDH system 200 of the present disclosure. When the ammonia production system 170 produces ammonia product, the ammonia product that is produced is fairly cold, for example around −32° C. However, in embodiments of the modified PDH system 200 that include the acrylonitrile system 11, the ammonia fed from the ammonia production system 170 to the acrylonitrile system 11 must be preheated before being sent to the acrylonitrile system 11, for example from about −32° C. to ambient temperature. Thus, in such embodiments, the cold ammonia produced in the ammonia production system 170 can be used as a heat sink in the ammonia refrigeration cycle of the ammonia synthesis loop 10. The resulting synergistic benefits to doing so are the elimination of a need for separate cooling water systems that would otherwise be used as a heat sink for the ammonia refrigeration cycle, achieving the required increase in the temperature of the ammonia before it enters the acrylonitrile system 11 without the need for an additional heater or heat exchanger to do so, and a reduction in overall energy use and dependency on utilities (e.g. electrical utilities).

For acrylonitrile plants, using the modified PDH system 200 of the present disclosure to produce acrylonitrile provides many technological advantages over using conventional ammoxidation of propane technologies that are currently used to produce acrylonitrile. First, production of acrylonitrile using the modified PDH system 200 has a propane utilization efficiency (defined as the mass of the valuable products produced from propane per unit mass of propane that is consumed as feed stock) of up to 88%, versus 49% for production of acrylonitrile by the ammoxidation of propane. Also, the modified PDH system 200 is a self-dependent process, in that it does not require the purchase of ammonia to produce acrylonitrile as does a conventional stand-alone acrylonitrile plant.

Included below are tables showing consumption and resulting product outputs for two exemplary embodiments of the modified PDH process of the present disclosure. However, it should be understood that such tables are included for exemplary illustration purposes only to show potential system capabilities of the modified PDH system, and are not to be read as limiting the scope of the present disclosure to any of the shown values in the tables. Alternate consumption and output values may be achieved by adjusting various operational parameters of the modified PDH system and/or including additional downstream systems. Further, in the below exemplary tables, “t/h” means tonnes per hour (or metric tons per hour), and “MMBtu/h” means million Btu per hour.

### Example 1

The below table shows the consumption and intermediary and/or end product output capabilities for an exemplary embodiment of a modified PDH system of the present disclosure, wherein excess propene is used to additionally make acrylic acid, and wherein 172,656 tons per anum (TPA) of acrylonitrile (AN) and 49,579 TPA of acrylic acid (ACA) are produced.

| | |
|---|---|
| Consumption | Products |

PDH Plant

| | | | |
|---|---|---|---|
| Propane required (t/h) | 33.0 | Propylene (t/h) | 27.8 |
| Fuel cons Reformers | 467.5 | Hydrogen (kmol/h) | 967.0 |
| (MMBtu/h) | | | |
| Fuel cons HP boiler | 275 | Process condensate (t/h) | 84.2 |
| (MMBtu/h) | | | |
| BFW cons HP boiler (t/h) | 125 | BDO (t/h) | 2.5 |
| Cooling water (t/h) | 24,740.9 | | |

Ammonia Plant

| | | | |
|---|---|---|---|
| Hydrogen required (kmol/h) | 967.0 | Ammonia (t/h) | 10.9 |
| Nitrogen required (kmol/h) | 322.3 | Steam Export (t/h) | 8.2 |
| CW cons. (t/h) | 426.2 | BDO Export (kg/h) | 171.9 |
| BFW cons. (t/h) | 8.4 | | |
| Electrical consumption | 25.0 | | |
| (PDH + Ammonia) (MW) | | | |

Acrylonitrile Plant

| | | | |
|---|---|---|---|
| Propylene required (t/h) | 22.5 | ACN (t/h) | 21.8 |
| Ammonia required (t/h) | 10.9 | Amm.Sulfate (t/h) | 11.6 |
| Sulfuric acid con. (t/h) | 7.8 | Crude HCN (t/h) | 2.8 |
| Catalyst losses (kg/h) | 12.0 | Crude Acetonitrile (t/h) | 1.5 |
| CW cons. (t/h) | 5474.5 | Steam Export (t/h) | 41.8 |
| BFW cons.(t/h) | 181.5 | BDO Export (t/h) | 3.6 |
| | | Process Condensate (t/h) | 136.1 |
| | | Cont.water to be | 39.2 |
| | | treated (t/h) | |

Acrylic Acid Plant

| | | | |
|---|---|---|---|
| Propylene required (t/h) | 5.2 | ACA (t/h) | 6.3 |
| Cooling water | 5,070.6 | Acetic acid (kg/h) | 370.0 |
| consumption (t/h) | | | |
| Demin water | 2.5 | Cont.water to be | 20.8 |
| consumption (t/h) | | treated (t/h) | |
| Process steam (t/h) | 17.88 | | |
| Process oxygen (kmol/h) | 280.9 | | |
| Chilled Water (t/h) | 6,210 | | |
| Reboiler steam (t/h) | 57.5 | | |

### Example 2

The below table shows the consumption and intermediary and/or end product output capabilities for an alternate embodiment of a modified PDH system 200 of the present disclosure. In this embodiment, all of the ammonia produced by the ammonia production system 170 from hydrogen recovered by the hydrogen recovery unit 7, as well as the propene 145 produced in the propene production system 157, to the acrylonitrile production system 11. The excess propene is sold/exported to an external customer. The design basis of this embodiment of a modified PDH system 200 is the production of 172,656 tons per anum (TPA) of acrylonitrile (AN) and 41,976 TPA of liquid propene.

| | |
|---|---|
| Consumption | Products |

PDH Plant

| | | | |
|---|---|---|---|
| Propane required (t/h) | 33.0 | Propylene (t/h) | 22.5 |
| Fuel cons Reformers | 467.5 | Hydrogen (kmol/h) | 967.0 |
| (MMBtu/h) | | | |
| Fuel cons HP boiler | 227.4 | Process condensate | 84.2 |
| (MMBtu/h) | | (t/h) | |
| BFW cons HP boiler (t/h) | 110 | BDO (t/h) | 2.2 |
| Cooling water (t/h) | 24,740.9 | Excess propylene (t/h) | 5.3 |

Ammonia Plant

| | | | |
|---|---|---|---|
| Hydrogen required (kmol/h) | 967.0 | Ammonia (t/h) | 10.9 |
| Nitrogen required (kmol/h) | 322.3 | Steam Export (t/h) | 8.2 |
| CW cons. (t/h) | 426.2 | BDO Export (kg/h) | 171.9 |
| BFW cons. (t/h) | 8.4 | | |
| Electrical consumption | 25.0 | | |
| (PDH + Ammonia) (MW) | | | |

Acrylonitrile Plant

| | | | |
|---|---|---|---|
| Propylene required (t/h) | 22.5 | ACN (t/h) | 21.8 |
| Ammonia required (t/h) | 10.9 | Amm.Sulfate (t/h) | 11.6 |
| Sulfuric acid con. (t/h) | 7.8 | Crude HCN (t/h) | 2.8 |
| Catalyst losses (kg/h) | 12.0 | Crude Acetonitrile (t/h) | 1.5 |
| CW cons. (t/h) | 5474.5 | Steam Export (t/h) | 41.8 |
| BFW cons.(t/h) | 181.5 | BDO Export (t/h) | 3.6 |
| | | Process Condensate | 136.1 |
| | | (t/h) | |
| | | Cont.water to be treated | 39.2 |
| | | (t/h) | |

What is claimed is:

1. A modified propane dehydrogenation (PDH) system for producing one or more chemical products from propane, comprising:
a PDH reactor configured to convert propane into a reactor output stream of process gas containing at least propene, hydrogen, and waste gas;
a de-ethanizer stripper system operatively coupled to and disposed downstream of said PDH reactor and configured to separate the reactor output stream of process gas into a first de-ethanizer output stream containing propene and any chemical component heavier than propene to be further purified downstream for use as an end product or intermediate product, and a second de-ethanizer output stream containing hydrogen and waste gas; and
a hydrogen recovery unit operatively coupled to and disposed downstream of said de-ethanizer stripper system that is configured to separate the second de-ethanizer output stream into a stream of waste gas, and into a stream of hydrogen as one of an end product or an intermediate product to be used as a feed stock by an additional downstream system,
wherein said modified PDH system generates a stream of carbon dioxide emissions as a byproduct of at least PDH side reactions.

2. The modified PDH system of claim 1, wherein said hydrogen recovery unit comprises a hydrogen purification device configured to purify the separated stream of hydrogen into ultra-high purity hydrogen.

3. The modified PDH system of claim 1, further comprising:
a cold box operatively coupled to and disposed downstream of said de-ethanizer stripper system and upstream of said hydrogen recovery unit, said cold box being configured to recover any residual propane and propene present in the stream of hydrogen and waste gas leaving the de-ethanizer stripper and send it back to the de-ethanizer stripper for additional processing, and further configured to send the stream of hydrogen and waste gas downstream to the hydrogen recovery unit.

4. The modified PDH system of claim 1, further comprising:
a C3 splitter system operatively coupled to and disposed downstream of said de-ethanizer stripper system, the C3 splitter system being configured to separate out a stream of high purity propene as an end or intermediate product from the stream of propene and any chemical component heavier than propene that exits the de-ethanizer stripper.

5. The modified PDH system of claim 1, further comprising:
an ammonia production system operatively coupled to and disposed downstream of said hydrogen recovery unit that is configured to use the stream of hydrogen from said hydrogen recovery unit as a feed stock to produce a stream of ammonia as one of an end or intermediate product.

6. The modified PDH system of claim 5, further comprising:
an acrylonitrile production system operatively coupled to and disposed downstream of said ammonia production system that is configured to use both the stream of propene, together with the stream of ammonia from said ammonia production system, as feed stock to produce acrylonitrile by propene ammoxidation as one of an end or intermediate product.

7. The modified PDH system of claim 5, further comprising:
a urea production system operatively coupled to and disposed downstream of each of said ammonia production system and said fuel system, and configured to use both the stream of ammonia from said ammonia production system and the stream of carbon dioxide emissions, as feed stock to produce urea as one of an end or intermediate product.

8. The modified PDH system of claim 1, further comprising:
a methanol production system operatively coupled to and disposed downstream of each of said hydrogen recovery unit and said fuel system, and configured to use both the stream of hydrogen from said hydrogen recovery unit and the stream of carbon dioxide emissions, as feed stock to produce methanol as one of an end or intermediate product.

9. A modified PDH system for producing a chemical product from propene and hydrogen, the system comprising:
a first system configured to produce an output stream of propene by propane dehydrogenation (PDH);
a second system operatively coupled to said first system and requiring propene and hydrogen as feed stock to produce the chemical product; and
a hydrogen recovery unit disposed in said first system and operatively coupled to said second system, said hydrogen recovery unit being configured to recover and purify hydrogen from said first system and provide a stream of purified hydrogen from said first system to one of said second system or a third system disposed downstream.

10. The modified PDH system of claim 9, wherein said first system is configured to produce propene and hydrogen, and wherein said second system is configured to produce one or more of ammonia, acrylonitrile, urea, or methanol concurrently with the production of the propene and hydrogen.

11. A method for producing one or more chemical products from propane by a modified propane dehydrogenation (PDH) process, comprising:
reacting propane by a PDH process to produce a reactor output stream of process gas containing at least propene, hydrogen, and waste gas;
in a de-ethanizer stripper system, separating the reactor output stream of process gas into a first output stream containing propene and any chemical component heavier than propene, and a second output stream containing hydrogen and waste gas;
separating the first output stream into a stream of propene and a separate stream of chemical components heavier than propene, the stream of propene being one of an end product or an intermediate product to be used by an additional downstream system;
in a hydrogen recovery unit, recovering hydrogen from the second output stream to generate a stream of recovered hydrogen and a separate stream of waste gas, the stream of hydrogen being one of either an end product or an intermediate product to be used as a feed stock for an additional downstream system.

12. The method of claim 11, wherein said step of recovering hydrogen further comprises purifying the stream of recovered hydrogen to generate a purified hydrogen stream that is one of an end product, or an intermediate product to be used by an additional downstream system.

13. The method of claim 11, wherein said step of recovering hydrogen is performed downstream of said step of separating the first output stream into a stream of propene and a separate stream of chemical components heavier than propene.

14. The method of claim 11, further comprising:
reacting the stream of recovered hydrogen in an ammonia production system to produce an ammonia stream simultaneously with the propene stream, the ammonia stream being one of either an end product or an intermediate product to be used as a feed stock for an additional downstream system.

15. The method of claim 14, further comprising:
splitting the stream of propene into a partial stream of propene and an excess stream of propene; and
reacting the stream of ammonia produced by the ammonia production system with the partial the stream of propene in an acrylonitrile system to produce acrylonitrile as an end product by propene ammoxidation, simultaneously with the production of the excess propene stream as an end product.

16. The method of claim 14, further comprising:
generating a stream of carbon dioxide emissions from operation of the modified PDH process; and
reacting the stream of ammonia from the ammonia production system with the stream of carbon dioxide emissions in a urea production system to produce urea as an end product, simultaneously with the production of the propene stream as an end product.

17. The method of claim 11, further comprising:
generating a stream of carbon dioxide emissions from operation of the modified PDH process; and
reacting the stream of recovered hydrogen from the hydrogen recovery unit with the stream of carbon dioxide emissions in a methanol production system to produce methanol as an end product, simultaneously with the production of the propene stream as an end product.

18. The method of claim 11, wherein said hydrogen recovery unit recovers at least 99.95% hydrogen from the second output stream from the de-ethanizer stripper system.

19. The method of claim 11, wherein the stream of propene and the stream of recovered hydrogen are used to produce at least one of ammonia, acrylonitrile, urea, or methanol concurrently with the stream of propene and the stream of recovered hydrogen.
